(19) United States
(12) Patent Application Publication
(10) Pub. No.: US 2022/0145391 A1
(43) Pub. Date: May 12, 2022

(54) ASSESSING RISK WITH TOTAL CELL-FREE DNA

(71) Applicant: The Medical College of Wisconsin, Inc., Milwaukee, WI (US)

(72) Inventors: Aoy Tomita Mitchell, Elm Grove, WI (US); Michael Mitchell, Elm Grove, WI (US)

(73) Assignee: The Medical College of Wisconsin, Inc., Milwaukee, WI (US)

(21) Appl. No.: 17/349,041

(22) Filed: Jun. 16, 2021

Related U.S. Application Data
(63) Continuation of application No. PCT/US2019/066974, filed on Dec. 17, 2019.
(60) Provisional application No. 62/780,472, filed on Dec. 17, 2018.

(51) Int. Cl. C12Q 1/6883

(52) U.S. Cl. CPC C12Q 1/6883; C12Q 2600/158; C12Q 2600/106; C12Q 2600/118

(57) ABSTRACT

This invention relates to methods and compositions for assessing amount(s) of total cell-free DNA in a subject. Such amount(s) can be used to determine risk in a subject, which subjects are non-transplant subjects, such as surgical subjects, in some preferred embodiments.

### RELATED APPLICATION

This application claims the benefit of priority under 35 U.S.C. § 119 to U.S. Provisional Application No. 62/780,472, filed Dec. 17, 2018, the entire contents of which are incorporated herein by reference.

### FIELD OF THE INVENTION

This invention relates to methods and related compositions for assessing a total amount of cell-free nucleic acids, such as cell-free DNA (cf-DNA), in a sample from a subject. The methods and compositions provided herein can be used to determine risk in a subject, preferably non-transplant subjects, in some embodiments.

### SUMMARY OF INVENTION

The present disclosure is based, at least in part on the surprising discovery that total cf-DNA can be a marker of risk for a number of conditions, including non-transplant conditions, inflammatory conditions, in a subject. It also has been surprisingly found that total cf-DNA can be used as a marker to assess organ injury, stress and/or dysfunction. Finally, it has also been found that total cf-DNA can be used as a marker to monitor the effectiveness in subjects undergoing treatment for a condition, such as any one of the conditions provided herein.

Therefore, obtaining amount(s) of total cf-DNA are provided herein whereby the amount(s) can be used to assess the general health of a subject and/or assess any one of the foregoing and including therapeutic efficacy, infection, inflammation or inflammatory processes, cellular or tissue injury, etc. Any one of the methods provided herein can be used for such purposes.

Provided herein are methods related to obtaining amount(s) of total cf-DNA at one or more points in time. Also provided are related reports, kits, databases, compositions, etc. related to such determinations and/or including such amount(s) alone in combination with threshold value(s) or other amount(s), such as other amount(s) obtained from other points in time. What is provided herein are ways and related aspects of monitoring the health of a subject over time with total cf-DNA as a biomarker, including in non-transplant subjects.

In any one of the embodiments of any one of the methods provided herein, the subject may be any of the subjects provided herein, such as a subject that has undergone surgery such as cardiac surgery.

In any one of the embodiments of any one of the methods provided herein, where the subject is a transplant recipient, the subject has received an autograft or has received a transplanted organ or more than one organ, such as a heart and lung transplant.

In any one of the embodiments of any one of the methods provided herein, the threshold is any one of the thresholds provided herein.

### BRIEF DESCRIPTION OF FIGURES

The accompanying figures are not intended to be drawn to scale. The figures are illustrative only and are not required for enablement of the disclosure.

FIG. 1 provides an exemplary, non-limiting diagram of MOMA primers. In a polymerase chain reaction (PCR) assay, extension of the sequence containing SNV A is expected to occur, resulting in the detection of SNV A, which may be subsequently quantified. Extension of the SNV B; however, is not expected to occur due to the double mismatch.

FIG. 2 illustrates an example of a computer system with which some embodiments may operate.

FIG. 3 is a graph showing the experimental determination of a cutpoint (threshold) for death using total cf-DNA from 298 samples.

FIG. 4 shows the experimental determination of a cutpoint (threshold) for death using total cf-DNA from the 85 samples.

FIG. 5 shows the experimental determination of a cutpoint (threshold) for cardiac arrest using total cf-DNA from the 85 samples.

FIG. 6 shows the experimental determination of a cutpoint (threshold) for infection (i.e., whether the subject was undergoing treatment for infection at the time of the sample) using total cf-DNA from the 292 samples.

FIG. 7 is a series of graphs depicting peak total cf-DNA levels and certain conditions. There was no difference based on gender or age; however, statistically significant associations between peak TCF (total cell free DNA) levels at any time post operatively and cardiac arrest (C.arrest), prolonged ventilation, death, prolonged length of stay (LOS), infection, and mechanical circulatory support were found. N (patient numbers) are included as well as p value (two-sided t-test).

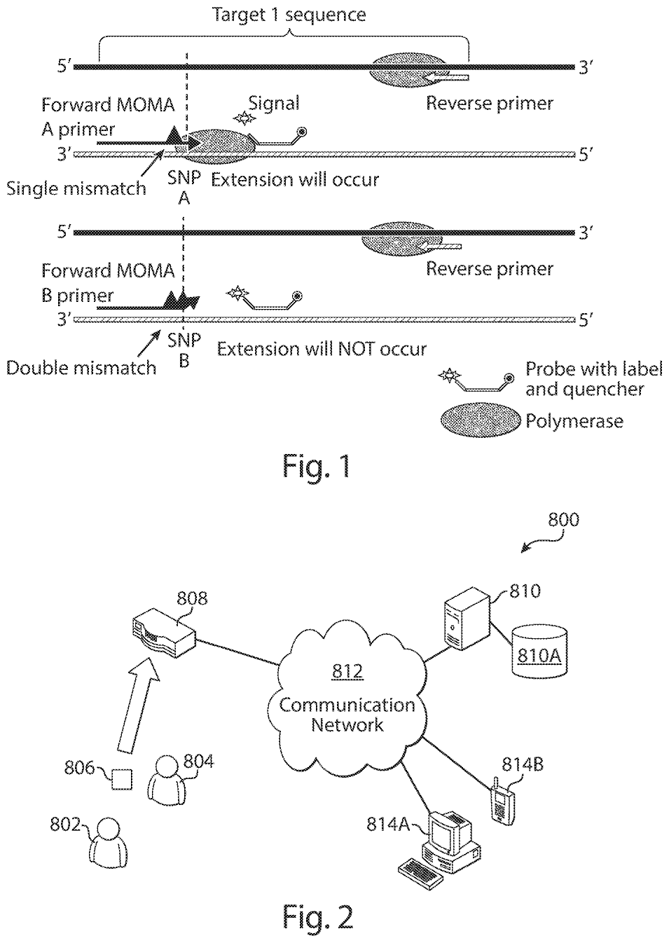

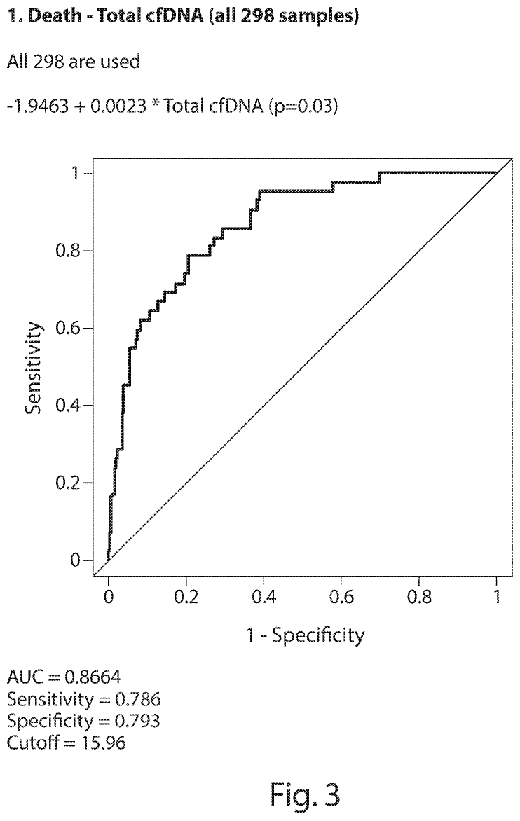

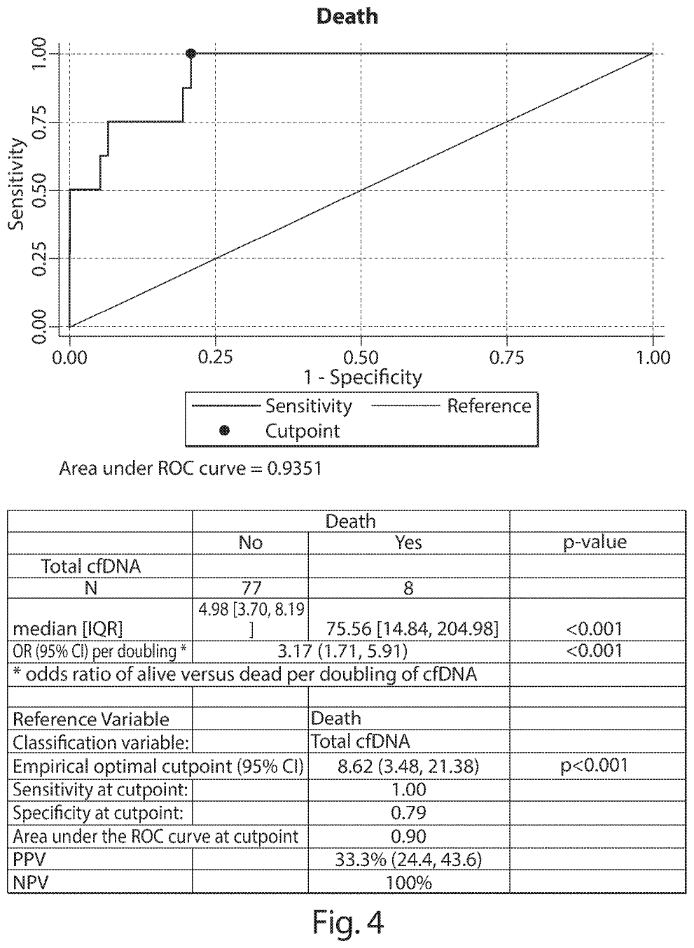

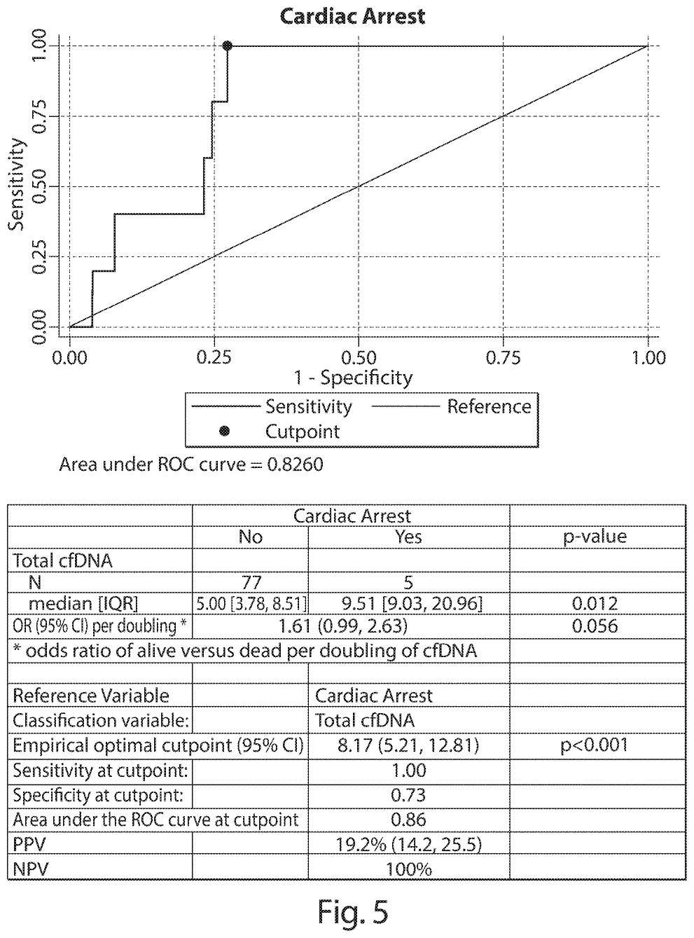

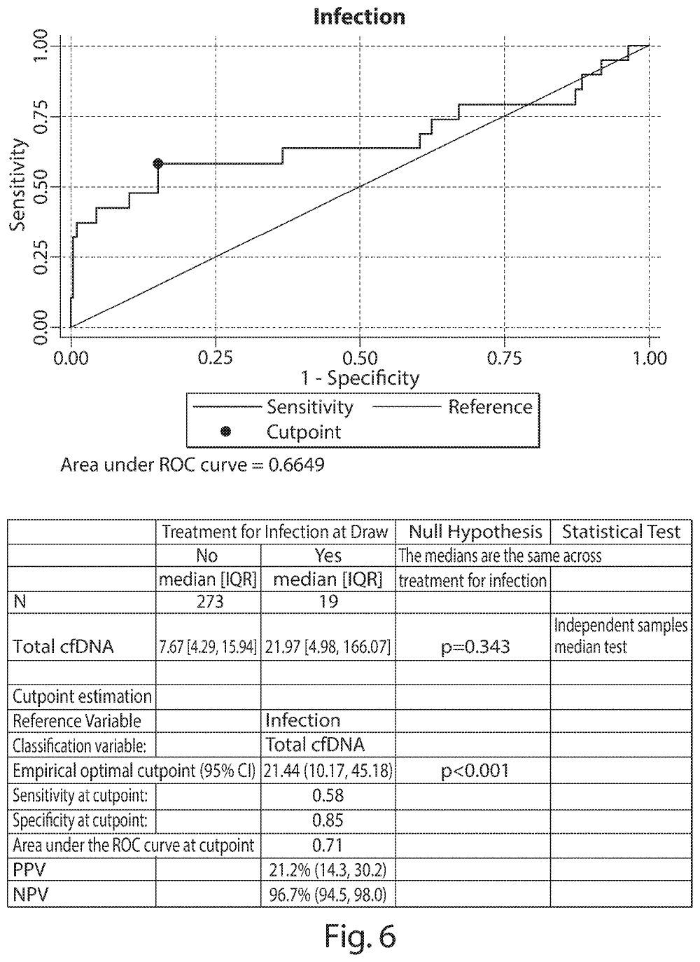

### DETAILED DESCRIPTION OF THE INVENTION

Aspects of the disclosure relate to methods of quantifying total cell-free DNA (such as in ng/ml) in a sample in order to determine a risk in a subject, such as any one of those provided herein. As provided herein, early detection or monitoring of the state or condition of a subject, such as one with any one of the conditions provided herein or one that has had surgery, such as non-transplant surgery, such as heart surgery (e.g., cardiopulmonary bypass) can facilitate treatment and/or monitoring and improve clinical outcomes. In some embodiments, the subject may show no signs or symptoms of the state or condition or risk associated therewith. However, in some embodiments, the subject may show symptoms.

As an example, post-surgical complications are a major cause of prolonged hospital stays and late mortality. Treatment of post-surgical complications with an appropriate therapy has been shown to improve surgical treatment outcomes, particularly if the complication is detected early. Following surgery, subjects are monitored for surgery-specific complications. As an example, after cardiac surgery, routine monitoring includes: continuous telemetry, measurement of the arterial blood pressure via an arterial catheter, measurement of the cardiac filling pressures via a pulmonary artery catheter (i.e., right heart catheter, Swan Ganz catheter), continuous assessment of the arterial oxygen saturation via pulse oximetry, and the continuous measurement of the mixed venous oxygen saturation via an oximetric pulmonary artery catheter. The invasive procedures, however, are associated with risks and discomfort for a patient, and may be particularly disadvantageous for pediatric patients. Accordingly, provided herein are sensitive, specific, cost effective, and non-invasive techniques for the surveillance of subjects, such as surgical patients. Such techniques have been found to allow for the detection of undesirable states or conditions of a subject, even at an early stage. Such techniques can also be used to monitor subject recovery and in the selection and monitoring of a treatment or therapy, such as an anti-infection treatment, thus improving a subject's recovery and increasing survival rates.

Thus, in one embodiment of any one of the methods provided herein the subject is one that has undergone surgery. In one of such embodiments, the subject is a non-transplant subject (i.e., one that has not received a transplant) or a subject that has had a surgery in addition to a transplant surgery at the same or a previous time, the monitoring preferably, in such embodiments, being for the non-transplant surgery.

In another embodiment of any one of the methods provided herein, the subject is one that has or is suspected of having an infection or infectious disease. An “infection” or “infectious disease” is any condition or disease caused by a microorganism, pathogen or other agent, such as a bacterium, fungus, prion or virus.

Such a subject may be one that has been or is being administered an anti-infection treatment. Anti-infection treatments include therapies for treating a bacterial, fungal and/or viral infections. Such therapies include antibiotics. Other examples include, but are not limited to, amebicides, aminoglycosides, anthelmintics, antifungals, azole antifungals, echinocandins, polyenes, diarylquinolines, hydrazide derivatives, nicotinic acid derivatives, rifamycin derivatives, streptomyces derivatives, antiviral agents, chemokine receptor antagonist, integrase strand transfer inhibitor, neuraminidase inhibitors, NNRTIs, NS5A inhibitors, nucleoside reverse transcriptase inhibitors (NRTIs), protease inhibitors, purine nucleosides, carbapenems, cephalosporins, glycylcyclines, leprostatics, lincomycin derivatives, macrolide derivatives, ketolides, macrolides, oxazolidinone antibiotics, penicillins, beta-lactamase inhibitors, quinolones, sulfonamides, and tetracyclines. Other such therapies are known to those of ordinary skill in the art. Other anti-infection treatments are known to those of ordinary skill in the art.

In another embodiment of any one of the methods provided herein, the subject is one in which inflammation is occurring or one that has or is suspected of having an inflammatory disease or disorder. As used herein, an “inflammatory disease or disorder” is any one in which the disease or disorder occurs, or symptoms thereof, are at least in part due to inflammation or an inflammatory process. Examples of such diseases or disorders include Alzheimer's, ankylosing spondylitis, arthritis (e.g., osteoarthritis, rheumatoid arthritis (RA), psoriatic arthritis), asthma, atherosclerosis, Crohn's disease, colitis, dermatitis, diverticulitis, fibromyalgia, hepatitis, irritable bowel syndrome (IBS), systemic lupus erythematous (SLE), nephritis, Parkinson's disease, ulcerative colitis, etc.

Such a subject may be one that has been or is being administered an anti-inflammatory. Anti-inflammatories include aspirin, celecoxib, diclofenac, ibuprofen, indomethacin, naproxen, oxaprozin, piroxicam, etc. Anti-inflammatories also include corticosteroids.

In another embodiment of any one of the methods provided herein, the subject is one in which any organ injury, stress or dysfunction is occurring or is suspected of occurring. Such subjects include those with cardiac cardiomyopathy, congestive heart failure, congestive heart disease, ischemia, cardiomyopathy or other conditions of an organ, such as the heart. Such subjects also include subject with organ failure. Such subjects include heart failure, renal failure or hepatic failure. Subjects that fall within this category may also be one with pulmonary arterial hypertension. Such a subject may be one that has been or is being administered treatment for any one of the foregoing.

In another embodiment of any one of the methods provided herein, the subject is one with sepsis or shock or is suspected of having sepsis or shock. Such a subject may be one that has been or is being administered treatment for sepsis or shock.

Since total cf-DNA can be a biomarker for general health, the subject of any one of the methods provided herein may be any subject receiving a treatment, such as for a disease or condition. The amount(s) of total cf-DNA may be used to assess the effectiveness of the treatment. The treatment may be any one of the treatments provided herein or otherwise known in the art.

In an embodiment of any one of the methods provided herein, the subject may have or be suspected of having any one of the disease or conditions provided herein. In an embodiments of any one of the methods provided herein, including methods whereby the subject is so defined, the subject is also a non-transplant subject.

As used herein, “cell-free DNA” (or cf-DNA) is DNA that is present outside of a cell, e.g., in the blood, plasma, serum, etc. of a subject. Without wishing to be bound by any particular theory or mechanism, it is believed that cf-DNA is released from cells, e.g., via apoptosis of the cells. “Total cell-free DNA” (or total cf-DNA) is the total amount of cf-DNA present in a sample. As used herein, the compositions and methods provided herein can be used to determine an amount of total cell-free DNA and a subject's risk of complications associated with, or following, a procedure (e.g., a heart surgery). Examples of complications include, but are not limited to, death, cardiac arrest, prolonged ventilation, prolonged length of stay in the hospital, infection, and requirement of mechanical circulatory support.

An amount of total cf-DNA can be “obtained” by any one of the methods provided herein, and any obtaining step(s) can include any one of the methods incorporated herein by reference or otherwise provided herein. “Obtaining” as used herein refers to any method by which the respective information or materials can be acquired. Thus, the respective information can be acquired by experimental methods. An amount of cf-DNA (DS and/or total) may be determined with experimental techniques, such as those provided elsewhere herein or otherwise known in the art. Respective materials can be created, designed, etc. with various experimental or laboratory methods, in some embodiments. The respective information or materials can also be acquired by being given or provided with the information, such as in a report, or materials. Materials may be given or provided through commercial means (i.e. by purchasing), in some embodiments.

Because of the ability to determine amounts of nucleic acids, such as cf-DNA, and the correlation with health, conditions and/or outcomes in a subject, the methods provided herein can be used to assess subjects. Thus, a risk of improving or worsening can be determined in such subjects. A “risk” as provided herein, refers to the presence or absence or progression of any undesirable state or condition in a subject, or an increased likelihood of the presence or absence or progression of such a state or condition. As provided herein “increased risk” refers to the presence or progression of any undesirable state or condition in a subject or an increased likelihood of the presence or progression of such a state or condition. As provided herein, “decreased risk” refers to the absence of any undesirable state or condition or progression in a subject or a decreased likelihood of the presence or progression (or increased likelihood of the absence or nonprogression) of such a state or condition. The risk in any one of the methods provided herein may be the presence or progression of any one of the conditions or states provided herein.

In an embodiment of any one of the methods provided herein, the method can be used to assess treatment efficacy in any one of the subjects provided herein.

As provided herein, early detection or monitoring can facilitate treatment and improve clinical outcomes in the subjects as provided herein. Any one of the methods provided can be performed on any one of the subjects provided herein. Such methods can be used to monitor a subject over time, with or without treatment. Further, such methods can aid in the selection, administration and/or monitoring of a treatment or therapy. Accordingly, the methods provided herein can be used to determine a treatment or monitoring regimen. Any one of the methods provided herein can comprise steps of determining a treatment and/or monitoring regimen.

“Determining a treatment regimen”, as used herein, refers to the determination of a course of action for treatment of the subject. In one embodiment of any one of the methods provided herein, determining a treatment regimen includes determining an appropriate therapy or information regarding an appropriate therapy to provide to a subject. In some embodiments of any one of the methods provided herein, the determining includes providing an appropriate therapy or information regarding an appropriate therapy to a subject. As used herein, information regarding a treatment or therapy or monitoring may be provided in written form or electronic form. In some embodiments, the information may be provided as computer-readable instructions. In some embodiments, the information may be provided orally.

The therapies can be, for example, for treating any one of the conditions or states provided herein. Suitable therapies are provided or are known to those of ordinary skill in the art.

Administration of a treatment or therapy may be accomplished by any method known in the art (see, e.g., Harrison's Principle of Internal Medicine, McGraw Hill Inc.). Preferably, administration of a treatment or therapy occurs in a therapeutically effective amount. Administration may be local or systemic. Administration may be parenteral (e.g., intravenous, subcutaneous, or intradermal) or oral. Compositions for different routes of administration are known in the art (see, e.g., Remington's Pharmaceutical Sciences by E. W. Martin).

“Determining a monitoring regimen”, as used herein, refers to determining a course of action to monitor a state or condition in the subject over time. In one embodiment of any one of the methods provided herein, determining a monitoring regimen includes determining an appropriate course of action for determining the amount of total cf-DNA in the subject over time or at a subsequent point in time, or suggesting such monitoring to the subject. This can allow for the measurement of variations in a clinical state and/or permit calculation of normal values or baseline levels (as well as comparisons thereto). In some embodiments of any one of the methods provided herein determining a monitoring regimen includes determining the timing and/or frequency of obtaining samples from the subject and/or determining or obtaining an amount of total cf-DNA.

In some embodiments, amounts of total cf-DNA can be plotted over time. In some embodiments, threshold values for the points in time may also be plotted. For example, the threshold values can represent desirable or healthy values for the state or condition of a subject. Such plotting can be helpful to determine risk and/or to monitor a subject's progress. Such threshold values can be determined using data from a sufficient number of subjects. A comparison with a subject's cf-DNA levels to such threshold values over a period of time can be used to predict risk. Alternatively, whether or not total cf-DNA amounts increase or decrease over time in a subject can alone be used to predict risk and/or assess the state or condition of the subject.

Increasing levels of total cf-DNA can correlate with increased risk, thus, a clinician may determine that a subject should undergo more frequent sampling if the subject's total cf-DNA is found to increase between time points. If a subject is found to have decreasing levels of total cf-DNA between time points, a clinician may determine that less frequent sampling is sufficient. Additionally, if a subject does not show a decrease, the clinician may determine that additional testing and/or treatment and/or another type of treatment may be necessary. Steps of performing any one or more of the foregoing may be included in any one of the methods provided herein. Timing and/or frequency of monitoring may also be determined by a comparison to threshold values or other amount(s), such as those determined at other point(s) in time.

In some embodiments of any one of the methods provided herein, each amount and time point may be recorded in a report or in a database. Threshold values may also be recorded in a report or in a database.

Reports with any one or more of the values as provided herein are also provided in an aspect. Reports may be in oral, written (or hard copy) or electronic form, such as in a form that can be visualized or displayed. Preferably, the report provides the amount of total cf-DNA in a sample. In some embodiments, the report provides amounts of total cf-DNA in samples from a subject over time, and can further include corresponding threshold values in some embodiments.

In some embodiments, the amounts and/or threshold values are in or entered into a database. In one aspect, a database with such amounts and/or values is provided. From the amount(s), a clinician may assess the need for a treatment or monitoring of a subject. Accordingly, in any one of the methods provided herein, the method can include assessing the amount of total cf-DNA in the subject at more than one point in time. Such assessing can be performed with any one of the methods provided herein.

As used herein, “amount” refers to any quantitative value for the measurement and can be given in an absolute or relative amount. As used herein, the term “level” can be used instead of “amount” but is intended to refer to the same types of values.

In some embodiments, any one of the methods provided herein can comprise comparing an amount of total cf-DNA to a threshold value, or to one or more prior amounts, to identify a subject at increased or decreased risk. In some embodiments of any one of the methods provided herein, a subject having an increased amount of total cf-DNA compared to a threshold value, or to one or more prior amounts, is identified as being at increased risk. In some embodiments of any one of the methods provided herein, a subject having a decreased or similar amount of total cf-DNA compared to a threshold value, or to one or more prior amounts, is identified as being at decreased or not increased risk.

“Threshold” or “threshold value”, as used herein, refers to any predetermined level or range of levels that is indicative of the presence or absence or progression of a state or condition or the presence or absence of a risk associated therewith. The threshold values can take a variety of forms. It can be single cut-off value, such as a median or mean. It can be established based upon comparative groups, such as where the risk in one defined group is double the risk in another defined group. It can be a range, for example, where the tested population is divided equally (or unequally) into groups, such as a low-risk group, a medium-risk group and a high-risk group, or into quadrants, the lowest quadrant being subjects with the lowest risk and the highest quadrant being subjects with the highest risk. The threshold value can depend upon the particular population selected. For example, an apparently healthy population will have a different ‘normal’ range. As another example, a threshold value can be determined from baseline values before the presence of a state or condition or risk or after a course of treatment. Such a baseline can be indicative of a normal or other state in the subject not correlated with the risk or state or condition that is being tested for. In some embodiments, the threshold value can be a baseline value of the subject being tested. Accordingly, the predetermined values selected may take into account the category in which the subject falls. Appropriate ranges and categories can be selected with no more than routine experimentation by those of ordinary skill in the art. The threshold values can be used for comparisons to make treatment and/or monitoring decisions. The determination can be done based on any one of the comparisons as provided herein with or without other indicators of risk or the state or condition of the subject.

The threshold values provided herein can be used to determine a risk level to a subject, in an embodiment of any one of the methods provided herein. In some embodiments, the peak amount of total cf-DNA is measured. As used herein “peak amount” refers to the highest value of serial samples. Accordingly, if a peak amount of total cf-DNA is less than 50 ng/ml plasma, the subject is at low risk. “Low risk” as used herein, includes a subject that has a shorter length of hospital stay or a shorter ventilation time and/or no cardiac arrest, death or infection. If a peak amount of total cf-DNA is greater than 100 ng/ml plasma, then the subject is at a high risk. “High risk” as used herein, includes a subject that is likely to experience near-term cardiac arrest, death, infection, and/or the need for mechanical circulatory support. Subjects at high risk are also likely to require a longer length of stay in the hospital and/or longer time on a ventilator. In an embodiment of any one of the methods provided herein the threshold value is 50 or 100 ng/ml. In an embodiment of any one of such methods, a subject with a value greater than a threshold may then be selected for treatment and/or further monitoring as provided herein. In an embodiment of any one of such methods provided herein, the method includes a step of further monitoring or treatment of the subject.

In some embodiments, the total cf-DNA level indicates increased or decreased risk. As provided herein “increased risk” refers to the presence or progression of any undesirable condition or state in a subject or an increased likelihood of the presence or progression of such a condition or state. As provided herein, “decreased risk” refers to the absence of any undesirable condition or state or progression in a subject or a decreased likelihood of the presence or progression (or increased likelihood of the absence or non-progression) of such a condition or state. Subjects at increased risk are also likely to require a longer length of stay in the hospital and/or longer time on a ventilator. In an embodiment of any one of the methods provided herein the threshold value is 50 or 100 ng/ml. In an embodiment of any one of such methods, a subject with a value greater than a threshold may then be selected for treatment and/or further monitoring as provided herein. In an embodiment of any one of such methods provided herein, the method includes a step of further monitoring or treatment of the subject.

As described above, the level of total cf-DNA may be used as a marker for risk. In some embodiments, the level of total cf-DNA is used as a trend monitor, for example, to determine if a subject is improving (e.g., if the subject's risk is lessening). In some embodiments, the level of total cf-DNA is used as an indicator of absolute risk; that is, near-term risk of poor clinical outcome, condition or state.

The threshold values can also be used for comparisons to make treatment and/or monitoring decisions. For example, if the amount of total cf-DNA is equal to or greater than 50 or 100 ng/ml and/or increasing over time in any one of the methods provided herein, further monitoring and/or treatment may be indicated.

Accordingly, any one of the methods provided herein may further include an additional test(s) for assessing the subject, or a step of suggesting such further testing to the subject (or providing information about such further testing). The additional test(s) may be any one of the methods provided herein. The additional test(s) may be any one of the other methods provided herein or otherwise known in the art as appropriate.

The amount of total cf-DNA, may be determined by a number of methods. In some embodiments such a method is a sequencing-based method. For example, the total cf-DNA may be measured by analyzing the DNA of a sample to identify multiple loci, an allele of each of the loci may be determined, and informative loci may be selected based on the determined alleles. As used herein, “loci” refer to nucleotide positions in a nucleic acid, e.g., a nucleotide position on a chromosome or in a gene.

The DNA may be analyzed using any suitable next generation or high-throughput sequencing and/or genotyping technique. Examples of next generation and high-throughput sequencing and/or genotyping techniques include, but are not limited to, massively parallel signature sequencing, polony sequencing, 454 pyrosequencing, Illumina (Solexa) sequencing, SOLiD sequencing, ion semiconductor sequencing, DNA nanoball sequencing, heliscope single molecule sequencing, single molecule real time (SMRT) sequencing, MassARRAY®, and Digital Analysis of Selected Regions (DANSR™) (see, e.g., Stein R A (1 Sep. 2008). “Next-Generation Sequencing Update”. Genetic Engineering & Biotechnology News 28 (15); Quail, Michael; Smith, Miriam E; Coupland, Paul; Otto, Thomas D; Harris, Simon R; Connor, Thomas R; Bertoni, Anna; Swerdlow, Harold P; Gu, Yong (1 Jan. 2012). “A tale of three next generation sequencing platforms: comparison of Ion torrent, pacific biosciences and illumina MiSeq sequencers”. BMC Genomics 13 (1): 341; Liu, Lin; Li, Yinhu; Li, Siliang; Hu, Ni; He, Yimin; Pong, Ray; Lin, Danni; Lu, Lihua; Law, Maggie (1 Jan. 2012). “Comparison of Next-Generation Sequencing Systems”. Journal of Biomedicine and Biotechnology 2012: 1-11; Qualitative and quantitative genotyping using single base primer extension coupled with matrix-assisted laser desorption/ionization time-of-flight mass spectrometry (MassARRAY®). Methods Mol Biol. 2009; 578:307-43; Chu T, Bunce K, Hogge W A, Peters D G. A novel approach toward the challenge of accurately quantifying fetal DNA in maternal plasma. Prenat Diagn 2010; 30:1226-9; and Suzuki N, Kamataki A, Yamaki J, Homma Y. Characterization of circulating DNA in healthy human plasma. Clinica chimica acta; International Journal of Clinical Chemistry 2008; 387:55-8).

In one embodiment, any one of the methods for determining total cf-DNA may be any one of the methods of U.S. Publication No. 2015-0086477-A1, and such methods are incorporated herein by reference in their entirety.

An amount of cf-DNA may also be determined by a mismatch amplification-based assay, such as a MOMA assay. In one embodiment, any one of the methods for determining cf-DNA may be any one of the methods of PCT Publication No. WO 2016/176662 A1, and such methods are incorporated herein by reference in their entirety.

In some embodiments of any one of the methods provided herein, the amplification-based quantitative assay is any quantitative assay, such as whereby nucleic acids are amplified and the amounts of the nucleic acids can be determined. Such assays include those whereby nucleic acids are amplified with the MOMA primers as described herein and quantified, or other primers. Such assays also include simple amplification and detection, hybridization techniques, separation technologies, such as electrophoresis, next generation sequencing and the like.

In some embodiments of any one of the methods provided herein the PCR is quantitative PCR meaning that amounts of nucleic acids can be determined. Quantitative PCR include real-time PCR, digital PCR, TAQMAN™, etc. In some embodiments of any one of the methods provided herein the PCR is “real-time PCR”. Such PCR refers to a PCR reaction where the reaction kinetics can be monitored in the liquid phase while the amplification process is still proceeding. In contrast to conventional PCR, real-time PCR offers the ability to simultaneously detect or quantify in an amplification reaction in real time. Based on the increase of the fluorescence intensity from a specific dye, the concentration of the target can be determined even before the amplification reaches its plateau.

The use of multiple probes can expand the capability of single-probe real-time PCR. Multiplex real-time PCR uses multiple probe-based assays, in which each assay can have a specific probe labeled with a unique fluorescent dye, resulting in different observed colors for each assay. Real-time PCR instruments can discriminate between the fluorescence generated from different dyes. Different probes can be labeled with different dyes that each have unique emission spectra. Spectral signals are collected with discrete optics, passed through a series of filter sets, and collected by an array of detectors. Spectral overlap between dyes may be corrected by using pure dye spectra to deconvolute the experimental data by matrix algebra.

A probe may be useful for methods of the present disclosure, particularly for those methods that include a quantification step. Any one of the methods provided herein can include the use of a probe in the performance of the PCR assay(s), while any one of the compositions or kits provided herein can include one or more probes. In some embodiments of any one or more of the methods provided herein, the probe in one or more or all of the PCR quantification assays is on the same strand as the mismatch primer and not on the opposite strand. It has been found that in so incorporating the probe in a PCR reaction, additional allele specific discrimination can be provided.

As an example, a TAQMAN™ probe is a hydrolysis probe that has a FAM™ or VIC® dye label on the 5′ end, and minor groove binder (MGB) non-fluorescent quencher (NFQ) on the 3′ end. The TAQMAN™ probe principle generally relies on the 5′-3′ exonuclease activity of Taq® polymerase to cleave the dual-labeled TAQMAN™ probe during hybridization to a complementary probe-binding region and fluorophore-based detection. TAQMAN™ probes can increase the specificity of detection in quantitative measurements during the exponential stages of a quantitative PCR reaction.

PCR systems generally rely upon the detection and quantitation of fluorescent dyes or reporters, the signal of which increase in direct proportion to the amount of PCR product in a reaction. For example, in the simplest and most economical format, that reporter can be the double-stranded DNA-specific dye SYBR® Green (Molecular Probes). SYBR® Green is a dye that binds the minor groove of double-stranded DNA. When SYBR® Green dye binds to a double-stranded DNA, the fluorescence intensity increases. As more double-stranded amplicons are produced, SYBR® Green dye signal will increase.

In any one of the methods provided herein the PCR may be digital PCR. Digital PCR involves partitioning of diluted amplification products into a plurality of discrete test sites such that most of the discrete test sites comprise either zero or one amplification product. The amplification products are then analyzed to provide a representation of the frequency of the selected genomic regions of interest in a sample. Analysis of one amplification product per discrete test site results in a binary “yes-or-no” result for each discrete test site, allowing the selected genomic regions of interest to be quantified and the relative frequency of the selected genomic regions of interest in relation to one another be determined. In certain aspects, in addition to or as an alternative, multiple analyses may be performed using amplification products corresponding to genomic regions from predetermined regions. Results from the analysis of two or more predetermined regions can be used to quantify and determine the relative frequency of the number of amplification products. Using two or more predetermined regions to determine the frequency in a sample reduces a possibility of bias through, e.g., variations in amplification efficiency, which may not be readily apparent through a single detection assay. Methods for quantifying DNA using digital PCR are known in the art and have been previously described, for example in U.S. Patent Publication number US20140242582.

It should be appreciated that the PCR conditions provided herein may be modified or optimized to work in accordance with any one of the methods described herein. Typically, the PCR conditions are based on the enzyme used, the target template, and/or the primers. In some embodiments, one or more components of the PCR reaction is modified or optimized. Non-limiting examples of the components of a PCR reaction that may be optimized include the template DNA, the primers (e.g., forward primers and reverse primers), the deoxynucleotides (dNTPs), the polymerase, the magnesium concentration, the buffer, the probe (e.g., when performing real-time PCR), the buffer, and the reaction volume.

In any of the foregoing embodiments, any DNA polymerase (enzyme that catalyzes polymerization of DNA nucleotides into a DNA strand) may be utilized, including thermostable polymerases. Suitable polymerase enzymes will be known to those skilled in the art, and include E. coli DNA polymerase, Klenow fragment of E. coli DNA polymerase I, T7 DNA polymerase, T4 DNA polymerase, T5 DNA polymerase, Klenow class polymerases, Taq polymerase, Pfu DNA polymerase, Vent polymerase, bacteriophage 29, REDTaq™ Genomic DNA polymerase, or sequenase. Exemplary polymerases include, but are not limited to Bacillus stearothermophilus pol I, Thermus aquaticus (Taq) pol I, Pyrccoccus furiosus (Pfu), Pyrccoccus woesei (Pwo), Thermus flavus (Tfl), Thermus thermophilus (Tth), Thermus litoris (Tli) and Thermotoga maritime (Tma). These enzymes, modified versions of these enzymes, and combination of enzymes, are commercially available from vendors including Roche, Invitrogen, Qiagen, Stratagene, and Applied Biosystems. Representative enzymes include PHUSION® (New England Biolabs, Ipswich, Mass.), Hot MasterTaq™ (Eppendorf), PHUSION® Mpx (Finnzymes), PyroStart® (Fermentas), KOD (EMD Biosciences), Z-Taq (TAKARA), and CS3AC/LA (KlenTaq, University City, Mo.).

Salts and buffers include those familiar to those skilled in the art, including those comprising MgCl2, and Tris-HCl and KCl, respectively. Typically, 1.5-2.0 nM of magnesium is optimal for Taq DNA polymerase, however, the optimal magnesium concentration may depend on template, buffer, DNA and dNTPs as each has the potential to chelate magnesium. If the concentration of magnesium [Mg2+] is too low, a PCR product may not form. If the concentration of magnesium [Mg2+] is too high, undesired PCR products may be seen. In some embodiments the magnesium concentration may be optimized by supplementing magnesium concentration in 0.1 mM or 0.5 mM increments up to about 5 mM.

Buffers used in accordance with the disclosure may contain additives such as surfactants, dimethyl sulfoxide (DMSO), glycerol, bovine serum albumin (BSA) and polyethylene glycol (PEG), as well as others familiar to those skilled in the art. Nucleotides are generally deoxyribonucleoside triphosphates, such as deoxyadenosine triphosphate (dATP), deoxycytidine triphosphate (dCTP), deoxyguanosine triphosphate (dGTP), and deoxythymidine triphosphate (dTTP), which are also added to a reaction adequate amount for amplification of the target nucleic acid. In some embodiments, the concentration of one or more dNTPs (e.g., dATP, dCTP, dGTP, dTTP) is from about 10 μM to about 500 μM which may depend on the length and number of PCR products produced in a PCR reaction.

In some embodiments, the concentration of primers used in the PCR reaction may be modified or optimized. In some embodiments, the concentration of a primer (e.g., a forward or reverse primer) in a PCR reaction may be, for example, about 0.05 μM to about 1 μM. In particular embodiments, the concentration of each primer is about 1 nM to about 1 μM. It should be appreciated that the primers in accordance with the disclosure may be used at the same or different concentrations in a PCR reaction. For example, the forward primer of a primer pair may be used at a concentration of 0.5 μM and the reverse primer of the primer pair may be used at 0.1 μM. The concentration of the primer may be based on factors including, but not limited to, primer length, GC content, purity, mismatches with the target DNA or likelihood of forming primer dimers.

In some embodiments, the thermal profile of the PCR reaction is modified or optimized. Non-limiting examples of PCR thermal profile modifications include denaturation temperature and duration, annealing temperature and duration and extension time.

The temperature of the PCR reaction solutions may be sequentially cycled between a denaturing state, an annealing state, and an extension state for a predetermined number of cycles. The actual times and temperatures can be enzyme, primer, and target dependent. For any given reaction, denaturing states can range in certain embodiments from about 70° C. to about 100° C. In addition, the annealing temperature and time can influence the specificity and efficiency of primer binding to a particular locus within a target nucleic acid and may be important for particular PCR reactions. For any given reaction, annealing states can range in certain embodiments from about 20° C. to about 75° C. In some embodiments, the annealing state can be from about 46° C. to 64° C. In certain embodiments, the annealing state can be performed at room temperature (e.g., from about 20° C. to about 25° C.).

Extension temperature and time may also impact the allele product yield. For a given enzyme, extension states can range in certain embodiments from about 60° C. to about 75° C.

Quantification of the amounts of the alleles from a PCR assay can be performed as provided herein or as otherwise would be apparent to one of ordinary skill in the art. As an example, amplification traces are analyzed for consistency and robust quantification. Internal standards may be used to translate the cycle threshold to amount of input nucleic acids (e.g., DNA). The amounts of alleles can be computed as the mean of performant assays and can be adjusted for genotype.

Other methods for determining total cell-free DNA in the subject are known in the art. In some embodiments of any one of the methods provided herein, the total cell-free DNA is determined with TAQMAN™ Real-time PCR using RNase P as a target or one or more other appropriate targets.

Any one of the methods provided herein can comprise extracting nucleic acids, such as cell-free DNA, from a sample obtained from a subject. Such extraction can be done using any method known in the art or as otherwise provided herein (see, e.g., Current Protocols in Molecular Biology, latest edition, or the QIAamp circulating nucleic acid kit or other appropriate commercially available kits). An exemplary method for isolating cell-free DNA from blood is described. Blood containing an anti-coagulant such as EDTA or DTA is collected from a subject. The plasma, which contains cf-DNA, is separated from cells present in the blood (e.g., by centrifugation or filtering). An optional secondary separation may be performed to remove any remaining cells from the plasma (e.g., a second centrifugation or filtering step). The cf-DNA can then be extracted using any method known in the art, e.g., using a commercial kit such as those produced by Qiagen. Other exemplary methods for extracting cf-DNA are also known in the art (see, e.g., Cell-Free Plasma DNA as a Predictor of Outcome in Severe Sepsis and Septic Shock. Clin. Chem. 2008, v. 54, p. 1000-1007; Prediction of MYCN Amplification in Neuroblastoma Using Serum DNA and Real-Time Quantitative Polymerase Chain Reaction. JCO 2005, v. 23, p. 5205-5210; Circulating Nucleic Acids in Blood of Healthy Male and Female Donors. Clin. Chem. 2005, v. 51, p. 131′7-1319; Use of Magnetic Beads for Plasma Cell-free DNA Extraction: Toward Automation of Plasma DNA Analysis for Molecular Diagnostics. Clin. Chem. 2003, v. 49, p. 1953-1955; Chiu R W K, Poon L L M, Lau T K, Leung T N, Wong E M C, Lo Y M D. Effects of blood-processing protocols on fetal and total DNA quantification in maternal plasma. Clin Chem 2001; 47:1607-1613; and Swinkels et al. Effects of Blood-Processing Protocols on Cell-free DNA Quantification in Plasma. Clinical Chemistry, 2003, vol. 49, no. 3, 525-526).

In some embodiments of any one of the methods provided herein, a pre-amplification step is performed. An exemplary method of such a pre-amplification is as follows, and such a method can be included in any one of the methods provided herein. Approximately 15 ng of cell-free plasma DNA is amplified in a PCR using Q5 DNA polymerase with approximately 13 targets where pooled primers were at 4 uM total. Samples undergo approximately 25 cycles. Reactions are in 25 ul total. After amplification, samples can be cleaned up using several approaches including AMPURE bead cleanup, bead purification, or simply ExoSAP-IT™, or Zymo.

As used herein, the sample from a subject can be a biological sample. Examples of such biological samples include whole blood, plasma, serum, urine, etc. In some embodiments, addition of further nucleic acids, e.g., a standard, to the sample can be performed.

Various aspects of the present invention may be used alone, in combination, or in a variety of arrangements not specifically discussed in the embodiments described in the foregoing and are therefore not limited in their application to the details and arrangement of components set forth in the foregoing description or illustrated in the drawings. For example, aspects described in one embodiment may be combined in any manner with aspects described in other embodiments.

Also, embodiments of the invention may be implemented as one or more methods, of which an example has been provided. The acts performed as part of the method(s) may be ordered in any suitable way. Accordingly, embodiments may be constructed in which acts are performed in an order different from illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting. The following description provides examples of the methods provided herein.

### EXAMPLES

### Example 1—Total Cf-DNA and Inflammation and Organ Dysfunction

Total cf-DNA can be used to identify early presymptomatic inflammation and organ dysfunction, such as in children following cardiac surgery, to ultimately allow for early intervention and gauge the effectiveness of targeted treatment strategies. The methods provided herein can improve patient care, such as for the highest risk categories of the most significant cause of infant mortality related to birth defects, those with congenital heart disease (CHD) undergoing pediatric heart surgery. The methods provided herein can improve clinical outcomes by facilitating the pre-clinical recognition of severe illness and inflammation and allow for the early initiation of targeted treatment strategies and interventions in many categories of patients. Potential outcomes for subjects include death, cardiac arrest, need for mechanical circulatory support and infection, such as following surgery.

It has been found that pre-symptomatic elevations in plasma total cf-DNA concentration is correlated with near term death, cardiac arrest, the need for mechanical circulatory support and infection in patients followed longitudinally following pediatric heart transplantation. However, total cf-DNA has not been validated as a biomarker of severity of illness within a heterogeneous population of pediatric cardiac surgical patients.

### Methods

The subject population for this example is infants and children undergoing cardiac surgery utilizing CPB. Biosamples are collected at times before and after CPB and postoperatively corresponding with standard clinical laboratory draws. Physiologic measures are automatically archived at one-minute intervals. Biomarker, physiologic, organ, and outcome measures are tested for association using univariate and panel regression methods. It is expected that levels of total cf-DNA will be elevated post operatively and decline to a baseline level within 3-7 days as the child recovers. Any one of the methods can comprise obtaining amounts of total cf-DNA within the first week of a surgery, such as daily. Any one of the methods provided herein can comprise obtaining amount of total cf-DNA after 3, 4, 5, 6 or 7 days of surgery. Higher total cf-DNA levels at each time point can be associated with higher measures of organ dysfunction and morbidity.

The methods provided herein can inform care providers early which patients are at higher risk for death, cardiac arrest or other significant clinical events and can allow intensive care providers to trend the effectiveness of therapies. The methods provided herein can be used for the early presymptom targeting of therapy, and monitoring of the effectiveness of therapy non-invasively. In addition, methods can be used in the targeting and monitoring of therapy for all patients with illness resulting in inflammation and organ dysfunction.

Congenital heart disease is the most common birth affecting almost 1% of live births. Approximately 30% of affected children require surgery. Cardiac surgery utilizing CPB is associated with the development of the systemic inflammatory response syndrome (SIRS) with and altered organ perfusion and oxygen delivery. The development of SIRS following CPB is associated with prolonged mechanical ventilation, longer intensive care unit and hospital length of stay, and mortality. Exposure to the CPB circuit and pump results in a systemic inflammatory response. Many clinical efforts have been aimed at reducing the inflammatory response to CPB including minimizing CPB times, corticosteroids and miniaturized CPB circuit volumes. These efforts have been met with quantifiable success however the overall mortality for children undergoing cardiac surgery remains approximately 3% and is over 15% in higher risk categories of patients.

Measures employed with variable success to assess severity of illness predict outcomes following CPB include laboratory biomarkers (Lactate, C-Reactive Protein, and Procalcitonin) (1-4) and predictive scoring systems such as the Inotrope score (IS), Vasoactive inotrope score (VIS) and vasoactive ventilation renal score (VVR) (3, 5-8). Data from a study in pediatric cardiac transplantation demonstrates that plasma total cf-DNA levels are associated with near term critical morbidity and mortality.

Current methods for the non-invasive assessment of severity of illness in children cared for in the intensive care setting are marked by variable success with imperfect sensitivity and severely limited specificity. Total cf-DNA is released into the circulation at baseline levels due to normal cell turnover and apoptosis. Cf-DNA is cleared rapidly from the circulation via hepatic metabolism with a half-life of approximately 15 min. Levels of cf-DNA dramatically increase with tissue injury, fever and illness and then drop to baseline levels within minutes once recovery occurs or the source of injury is removed. It has been found that total cf-DNA quantification in samples obtained prior to transplantation was predictive of near term death, cardiac arrest, and infection with optimal cutpoints of 8.62, 8.17, and 21.44 ng/ml, respectively. The apparent predictive relationship to near term death, with an optimal cutpoint achieving a negative predictive value of 100% was particularly striking.

Analysis of total cf-DNA was performed. Biomarker, physiologic, organ, and outcome measures are analyzed for association using univariate and panel regression methods, linking cellular biomarkers, physiologic responses, organ function, and outcome. The clinical data include renal regional oxygenation, as measured by NIRS, concurrent with cfDNA sampling. The details of the study are provided below.

A single-site, prospective, observational study of patients with congenital heart disease, less than 18 years of age, weighing 3.0 kg or more, undergoing surgery with cardiopulmonary bypass (CPB) was undertaken. Patients having a pre-operative need for ECMO or other forms mechanical circulatory support were excluded from the study.

The purpose of the study was to develop a total cell-free DNA assay for early pre-symptomatic identification of inflammation and organ dysfunction.

Blood was drawn so long as a clinically-required indwelling vascular access could be used. Samples were drawn prior to (1) surgical skin incision, (2) immediately after CPB, and after initiation of CPB at (3) 12 hrs, (4) 24 hrs, (5) 48 hrs, (6) 72 hrs, (7) 120 hrs and (8) 168 hrs. Contingent draws included: (1) sample obtained before vascular access was removed (if removed prior to post surgery day 7); (2) for inpatients beyond surgery Day 7, blood samples obtained weekly for up to 4 weeks; and (3) for weight less than 5 kg, samples 5 and 7 were omitted. In an embodiment of any one of the methods provided herein the samples are obtained or the level of total cf-DNA determined any one or more of the aforementioned timepoints in a subject, such as any one of the subject described herein, such as a surgical subject including one that has undergone heart surgery. The any one or more of the aforementioned timepoints includes the set of timepoints described.

One hundred and twenty pediatric patients undergoing surgery with cardiopulmonary bypass were enrolled in the study. Approximately half of the patients were less than 1 year of age and half were greater than 1 year of age.

The primary outcomes of the study included: the combined event of mortality, cardiac arrest (CA) or mechanical support (MS); individual critical events of mortality, cardiac arrest (CA), and mechanical support (MS); and infection.

The secondary outcomes of the study included: duration of mechanical ventilation, hospital length of stay, and low cardiac output syndrome.

Outcomes were assessed and entered into a database blinded to TCF levels. TCF levels (in ng/ml plasma) were entered into the database as well. Upon unblinding, there was no difference in peak TCF levels based on gender or age.

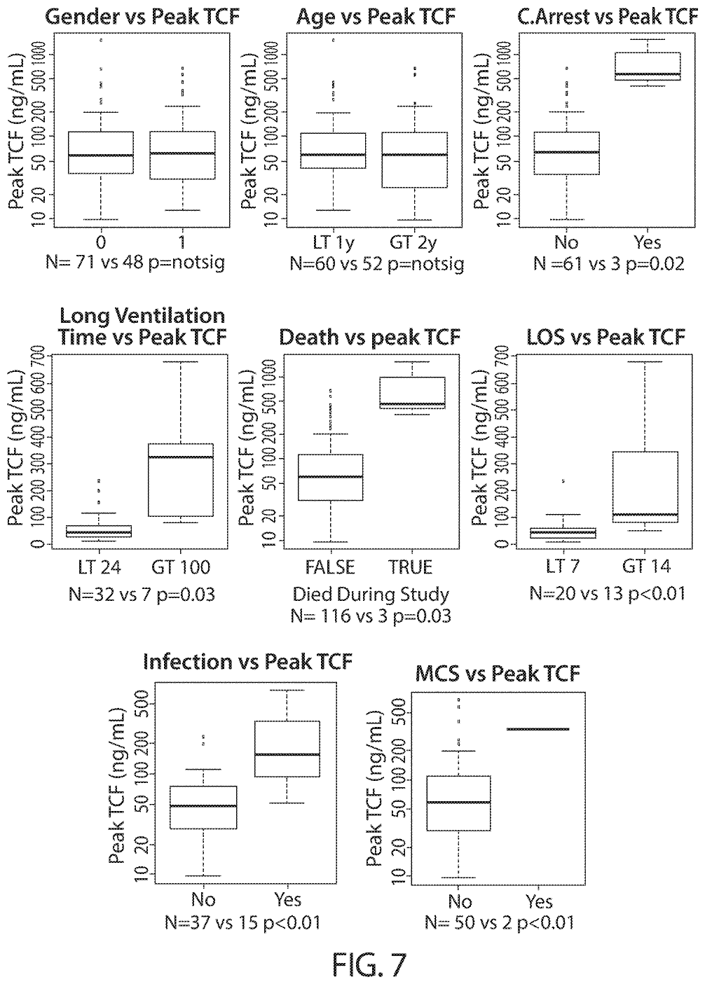

Summary statistics such as mean, median, standard deviation, range and correlation and plots were used to examine distributions and interrelationships. Exact binomial 95% confidence intervals were reported to enhance interpretation of the clinical relevance. To satisfy parametric assumptions, transformations or non-parametric analysis was used. However, statistically significant associations between peak TCF levels at any time post operatively and cardiac arrest, prolonged ventilation, death, prolonged length of stay, infection, and mechanical circulatory support were found, as shown in FIG. 7. Specifically, it was found that TCF (mg/ml plasma) peak levels less than 50 ng/ml were associated with low risk. Low TCF levels can be used as a biomarker for clinical risk, as a trend monitor, and as an indicator of absolute risk (e.g., near-term risk of poor clinical outcome). In contrast, TCF peak levels greater than 100 ng/ml plasma were found to be associated with high risk (e.g., prediction of near-term cardiac arrest, death, infection, and/or the need for mechanical circulatory support).

Accordingly, TCF plasma levels can be used to determine whether a patient is at high or low risk of poor clinical outcomes, conditions or states as provided herein.

### REFERENCES

1. Perez S B, Rodriguez-Fanjul J, Garcia L T, et al. Procalcitonin Is a Better Biomarker than C-Reactive Protein in Newborns Undergoing Cardiac Surgery: The PROKINECA Study. Biomark Insights. 2016; 11:123-9.
2. Sponholz C, Sakr Y, Reinhart K, et al. Diagnostic value and prognostic implications of serum procalcitonin after cardiac surgery: a systematic review of the literature. Crit Care. 2006; 10(5):R145.
3. Zant R, Stocker C, Schlapbach L J, et al. Procalcitonin in the Early Course Post Pediatric Cardiac Surgery. Pediatr Crit Care Med. 2016; 17(7):624-9.
4. Brocca A, Virzi G M, de Cal M, et al. Elevated Levels of Procalcitonin and Interleukin-6 are Linked with Postoperative Complications in Cardiac Surgery. Scand J Surg. 2017:1457496916683096.
5. Miletic K G, Delius R E, Walters H L, 3rd, et al. Prospective Validation of a Novel Vasoactive-Ventilation-Renal Score as a Predictor of Outcomes After Pediatric Cardiac Surgery. Ann Thorac Surg. 2016; 101(4):1558-63.
6. Davidson J, Tong S, Hancock H, et al. Prospective validation of the vasoactive-inotropic score and correlation to short-term outcomes in neonates and infants after cardiothoracic surgery. Intensive Care Med. 2012; 38(7):1184-90.
7. Delannoy B, Guye M L, Slaiman D H, et al. Effect of cardiopulmonary bypass on activated partial thromboplastin time waveform analysis, serum procalcitonin and C-reactive protein concentrations. Crit Care. 2009; 13(6):R180.
8. Butts R J, Scheurer M A, Atz A M, et al. Comparison of maximum vasoactive inotropic score and low cardiac output syndrome as markers of early postoperative outcomes after neonatal cardiac surgery. Pediatr Cardiol. 2012; 33(4):633-8.
9. Ahmed A I, Soliman R A, Samir S. Cell Free DNA and Procalcitonin as Early Markers of Complications in ICU Patients with Multiple Trauma and Major Surgery. Clin Lab. 2016; 62(12):2395-404.
10. Clementi A, Virzi G M, Brocca A, et al. The Role of Cell-Free Plasma DNA in Critically Ill Patients with Sepsis. Blood Purif. 2016; 41(1-3):34-40.
11. Purhonen A K, Juutilainen A, Vanska M, et al. Human plasma cell-free DNA as a predictor of infectious complications of neutropenic fever in hematological patients. Infect Dis (Lond). 2015; 47(4):255-9.
12. Avriel A, Paryente Wiessman M, Almog Y, et al. Admission cell free DNA levels predict 28-day mortality in patients with severe sepsis in intensive care. PLoS One. 2014; 9(6):e100514.
13. Dwivedi D J, Toltl L J, Swystun L L, et al. Prognostic utility and characterization of cell-free DNA in patients with severe sepsis. Crit Care. 2012; 16(4):R151.

### Example 2—Examples of Computer-Implemented Embodiments

In some embodiments, the techniques described above may be implemented via one or more computing devices executing one or more software facilities to analyze samples for a subject over time, measure nucleic acids (such as cell-free DNA) in the samples, and produce a diagnostic result based on one or more of the samples. FIG. 2 illustrates an example of a computer system with which some embodiments may operate, though it should be appreciated that embodiments are not limited to operating with a system of the type illustrated in FIG. 2.

The computer system of FIG. 2 includes a subject 802 and a clinician 804 that may obtain a sample 806 from the subject 806. As should be appreciated from the foregoing, the sample 806 may be any suitable sample of biological material for the subject 802 that may be used to measure the presence of nucleic acids (such as cell-free DNA) in the subject 802, including a blood sample. The sample 806 may be provided to an analysis device 808, which one of ordinary skill will appreciate from the foregoing will analyze the sample 808 so as to determine (including estimate) a total amount of nucleic acids (such as cell-free DNA) in the sample 806 and/or the subject 802. For ease of illustration, the analysis device 808 is depicted as single device, but it should be appreciated that analysis device 808 may take any suitable form and may, in some embodiments, be implemented as multiple devices. To determine the amounts of nucleic acids (such as cell-free DNA) in the sample 806 and/or subject 802, the analysis device 808 may perform any of the techniques described above, and is not limited to performing any particular analysis. The analysis device 808 may include one or more processors to execute an analysis facility implemented in software, which may drive the processor(s) to operate other hardware and receive the results of tasks performed by the other hardware to determine on overall result of the analysis, which may be the amounts of nucleic acids (such as cell-free DNA) in the sample 806 and/or the subject 802. The analysis facility may be stored in one or more computer-readable storage media, such as a memory of the device 808. In other embodiments, techniques described herein for analyzing a sample may be partially or entirely implemented in one or more special-purpose computer components such as Application Specific Integrated Circuits (ASICs), or through any other suitable form of computer component that may take the place of a software implementation.

In some embodiments, the clinician 804 may directly provide the sample 806 to the analysis device 808 and may operate the device 808 in addition to obtaining the sample 806 from the subject 802, while in other embodiments the device 808 may be located geographically remote from the clinician 804 and subject 802 and the sample 806 may need to be shipped or otherwise transferred to a location of the analysis device 808. The sample 806 may in some embodiments be provided to the analysis device 808 together with (e.g., input via any suitable interface) an identifier for the sample 806 and/or the subject 802, for a date and/or time at which the sample 806 was obtained, or other information describing or identifying the sample 806.

The analysis device 808 may in some embodiments be configured to provide a result of the analysis performed on the sample 806 to a computing device 810, which may include a data store 810A that may be implemented as a database or other suitable data store. The computing device 810 may in some embodiments be implemented as one or more servers, including as one or more physical and/or virtual machines of a distributed computing platform such as a cloud service provider. In other embodiments, the device 810 may be implemented as a desktop or laptop personal computer, a smart mobile phone, a tablet computer, a special-purpose hardware device, or other computing device.

In some embodiments, the analysis device 808 may communicate the result of its analysis to the device 810 via one or more wired and/or wireless, local and/or wide-area computer communication networks, including the Internet. The result of the analysis may be communicated using any suitable protocol and may be communicated together with the information describing or identifying the sample 806, such as an identifier for the sample 806 and/or subject 802 or a date and/or time the sample 806 was obtained.

The computing device 810 may include one or more processors to execute a diagnostic facility implemented in software, which may drive the processor(s) to perform diagnostic techniques described herein. The diagnostic facility may be stored in one or more computer-readable storage media, such as a memory of the device 810. In other embodiments, techniques described herein for analyzing a sample may be partially or entirely implemented in one or more special-purpose computer components such as Application Specific Integrated Circuits (ASICs), or through any other suitable form of computer component that may take the place of a software implementation.

The diagnostic facility may receive the result of the analysis and the information describing or identifying the sample 806 and may store that information in the data store 810A. The information may be stored in the data store 810A in association with other information for the subject 802, such as in a case that information regarding prior samples for the subject 802 was previously received and stored by the diagnostic facility. The information regarding multiple samples may be associated using a common identifier, such as an identifier for the subject 802. In some cases, the data store 810A may include information for multiple different subjects.

The diagnostic facility may also be operated to analyze results of the analysis of one or more samples 806 for a particular subject 802, identified by user input, so as to determine a diagnosis for the subject 802. The diagnosis may be a conclusion of a risk that the subject 802 has, may have, or may in the future develop a particular condition or state or such a condition or state may worsen or progress. The diagnostic facility may determine the diagnosis using any of the various examples described above, including by comparing the amounts of nucleic acids (such as cell-free DNA) determined for a particular sample 806 to one or more thresholds or by comparing a change over time in the amounts of nucleic acids (such as cell-free DNA) determined for samples 806 over time to one or more thresholds. For example, the diagnostic facility may determine a risk to the subject 802 of a condition by comparing a total amount of nucleic acids (such as cell-free DNA) for one or more samples 806 to one threshold and comparing a total amount of nucleic acids (such as cell-free DNA) for one or more different sample(s) to another threshold or amount at another point in time(s). Based on the comparisons to the thresholds, the diagnostic facility may produce an output indicative of a risk to the subject 802.

As should be appreciated from the foregoing, in some embodiments, the diagnostic facility may be configured with different thresholds or other amounts to which amounts of nucleic acids (such as cell-free DNA) may be compared. The different thresholds may, for example, correspond to different demographic groups (age, gender, race, economic class, presence or absence of a particular procedure/condition/other in medical history, or other demographic categories), different conditions, and/or other parameters or combinations of parameters. In such embodiments, the diagnostic facility may be configured to select thresholds or other amounts against which amounts of nucleic acids (such as cell-free DNA) are to be compared, with different thresholds or other amounts stored in memory of the computing device 810. The selection may thus be based on demographic information for the subject 802 in embodiments in which thresholds differ based on demographic group, and in these cases demographic information for the subject 802 may be provided to the diagnostic facility or retrieved (from another computing device, or a data store that may be the same or different from the data store 810A, or from any other suitable source) by the diagnostic facility using an identifier for the subject 802. The selection may additionally or alternatively be based on the condition or state for which a risk is to be determined, and the diagnostic facility may prior to determining the risk receive as input a condition and use the condition or state to select the thresholds or other amounts on which to base the determination of risk. It should be appreciated that the diagnostic facility is not limited to selecting thresholds or other amounts in any particular manner, in embodiments in which multiple thresholds or other amounts are supported.

In some embodiments, the diagnostic facility may be configured to output for presentation to a user a user interface that includes a diagnosis of a risk and/or a basis for the diagnosis for a subject 802. The basis for the diagnosis may include, for example, amounts of nucleic acids (such as cell-free DNA) detected in one or more samples 806 for a subject 802. In some embodiments, user interfaces may include any of the examples of results, values, amounts, graphs, etc. discussed above. They can include results, values, amounts, etc. over time. In such a case, in some cases the graph may be annotated to indicate to a user how different regions of the graph may correspond to different diagnoses that may be produced from an analysis of data displayed in the graph. For example, thresholds or other amounts against which the graphed data may be compared to determine the analysis may be imposed on the graph(s).

A user interface including a graph, particularly with the lines and/or shading, may provide a user with a far more intuitive and faster-to-review interface to determine a risk of the subject 802 based on amounts of nucleic acids (such as cell-free DNA), than may be provided through other user interfaces. It should be appreciated, however, that embodiments are not limited to being implemented with any particular user interface.

In some embodiments, the diagnostic facility may output the diagnosis or a user interface to one or more other computing devices 814 (including devices 814A, 814B) that may be operated by the subject 802 and/or a clinician, which may be the clinician 804 or another clinician. The diagnostic facility may transmit the diagnosis and/or user interface to the device 814 via the network(s) 812.

Techniques operating according to the principles described herein may be implemented in any suitable manner. Included in the discussion above are a series of flow charts showing the steps and acts of various processes that determine a risk of a condition based on an analysis of amounts of nucleic acids (such as cell-free DNA). The processing and decision blocks discussed above represent steps and acts that may be included in algorithms that carry out these various processes. Algorithms derived from these processes may be implemented as software integrated with and directing the operation of one or more single- or multi-purpose processors, may be implemented as functionally-equivalent circuits such as a Digital Signal Processing (DSP) circuit or an Application-Specific Integrated Circuit (ASIC), or may be implemented in any other suitable manner. It should be appreciated that embodiments are not limited to any particular syntax or operation of any particular circuit or of any particular programming language or type of programming language. Rather, one skilled in the art may use the description above to fabricate circuits or to implement computer software algorithms to perform the processing of a particular apparatus carrying out the types of techniques described herein. It should also be appreciated that, unless otherwise indicated herein, the particular sequence of steps and/or acts described above is merely illustrative of the algorithms that may be implemented and can be varied in implementations and embodiments of the principles described herein.

Accordingly, in some embodiments, the techniques described herein may be embodied in computer-executable instructions implemented as software, including as application software, system software, firmware, middleware, embedded code, or any other suitable type of computer code. Such computer-executable instructions may be written using any of a number of suitable programming languages and/or programming or scripting tools, and also may be compiled as executable machine language code or intermediate code that is executed on a framework or virtual machine.

When techniques described herein are embodied as computer-executable instructions, these computer-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include routines, programs, objects, components, data structures, etc. that perform particular tasks or implement particular abstract data types. Typically, the functionality of the functional facilities may be combined or distributed as desired in the systems in which they operate. In some implementations, one or more functional facilities carrying out techniques herein may together form a complete software package. These functional facilities may, in alternative embodiments, be adapted to interact with other, unrelated functional facilities and/or processes, to implement a software program application.

Some exemplary functional facilities have been described herein for carrying out one or more tasks. It should be appreciated, though, that the functional facilities and division of tasks described is merely illustrative of the type of functional facilities that may implement the exemplary techniques described herein, and that embodiments are not limited to being implemented in any specific number, division, or type of functional facilities. In some implementations, all functionality may be implemented in a single functional facility. It should also be appreciated that, in some implementations, some of the functional facilities described herein may be implemented together with or separately from others (i.e., as a single unit or separate units), or some of these functional facilities may not be implemented.

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as a Compact Disk (CD) or a Digital Versatile Disk (DVD), a persistent or non-persistent solid-state memory (e.g., Flash memory, Magnetic RAM, etc.), or any other suitable storage media. Such a computer-readable medium may be implemented in any suitable manner, including as a portion of a computing device or as a stand-alone, separate storage medium. As used herein, “computer-readable media” (also called “computer-readable storage media”) refers to tangible storage media. Tangible storage media are non-transitory and have at least one physical, structural component. In a “computer-readable medium,” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

In some, but not all, implementations in which the techniques may be embodied as computer-executable instructions, these instructions may be executed on one or more suitable computing device(s) operating in any suitable computer system, including the exemplary computer system of FIG. 2, or one or more computing devices (or one or more processors of one or more computing devices) may be programmed to execute the computer-executable instructions. A computing device or processor may be programmed to execute instructions when the instructions are stored in a manner accessible to the computing device or processor, such as in a data store (e.g., an on-chip cache or instruction register, a computer-readable storage medium accessible via a bus, etc.). Functional facilities comprising these computer-executable instructions may be integrated with and direct the operation of a single multi-purpose programmable digital computing device, a coordinated system of two or more multi-purpose computing device sharing processing power and jointly carrying out the techniques described herein, a single computing device or coordinated system of computing device (co-located or geographically distributed) dedicated to executing the techniques described herein, one or more Field-Programmable Gate Arrays (FPGAs) for carrying out the techniques described herein, or any other suitable system.

Embodiments have been described where the techniques are implemented in circuitry and/or computer-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments. Any one of the aforementioned, including the aforementioned devices, systems, embodiments, methods, techniques, algorithms, media, hardware, software, interfaces, processors, displays, networks, inputs, outputs or any combination thereof are provided herein in other aspects.

1-50. (canceled)

51. A method of assessing a risk in a subject, the method comprising:
(a) obtaining an amount of total cell-free DNA (cf-DNA) in a sample from the subject; and
(b) reporting and/or recording the amount of total cf-DNA.

52. The method of claim 51, wherein the method further comprises:
(c) comparing the amount of total cf-DNA to a threshold total cf-DNA value or other amount from a different point in time.

53. The method of claim 51, wherein the method further comprises:
(d) determining a risk in the subject based on the obtained amount of total cf-DNA or based on a comparison of a threshold total cf-DNA value or other total cf-DNA value from a different point in time.

54. The method of claim 51, wherein the method further comprises:
(e) obtaining an amount of total cf-DNA in one or more additional samples from the subject, each taken at different point in time.

55. The method of claim 54, wherein the method further comprises:
(f) comparing the amount(s) of total cf-DNA to threshold values or amounts from one or more prior points in time.

56. The method of claim 55, wherein the method further comprises:
(g) determining a risk in the subject based on a comparison(s) of (f).

57. The method of claim 51, wherein the method further comprises:
(h) determining a treatment or monitoring regimen for the subject based on the amounts of total cf-DNA and/or the comparison(s).

58. The method of claim 51, wherein total cf-DNA is obtained from samples taken from the subject on a daily, monthly or bimonthly basis.

59. The method of claim 51, wherein the subject is assessed for at least 1, 2 or 3 weeks or at least 1, 2, 3, 4, 5, 6, 9 or 12 months.

60. The method of claim 52, wherein the method further comprises providing one or more threshold values or amount from one or more different points in time.

61. The method of claim 51, wherein the method further comprises obtaining the sample(s) from the subject.

62. A report that comprises the amount(s) and/or threshold value(s) of claim 52.

63. A database that comprises the amount(s) and/or threshold value(s) of claim 52.

64. The method of claim 52, wherein an amount of total cf-DNA that is greater than a threshold value and/or is increased or increasing relative to amount(s) from earlier time point(s) represents an increased or increasing risk; and wherein an amount of total cf-DNA that is lower than a threshold value and/or is decreased or decreasing relative to the amount(s) from earlier time point(s) represents a decreased or decreasing risk.

65. The method of claim 54, wherein the time between samples is decreased if the amount of total cf-DNA is increased relative to threshold(s) or amount(s) from earlier time point(s), and wherein the time between samples is increased if the amount of total cf-DNA is decreased relative to threshold(s) or amount(s) from earlier time point(s).

66. The method of claim 57, wherein the determining a treatment regimen comprises treating the subject.

67. The method of claim 51, wherein:
(a) the subject is a surgical subject,
(b) the subject has or is suspected of having an infection,
(c) the subject has or is suspected of having an inflammatory disease or condition,
(d) the subject has or is suspected of having sepsis,
(e) the subject is in or is suspected of being in shock,
(f) the subject has or is suspected of having an organ injury, stress, dysfunction or failure,
(g) the subject has or is suspected of having pulmonary arterial hypertension;
(h) the subject is receiving or has received treatment for a condition; or
(i) the subject is, has, or is suspected of having any combination of (a)-(j).

68. The method of claim 51, wherein the amount(s) of total cf-DNA is obtained using an amplification-based quantification assay.

69. The method of claim 51, wherein the risk is increased or increasing if the amount(s) of total cf-DNA is greater than a threshold(s) or amount(s) from earlier time point(s); and wherein the risk is decreased or decreasing if the amount(s) of total cf-DNA is less than a threshold(s) or amount(s) from earlier time point(s).

70. The method of claim 52, wherein the threshold is 50 or 100 ng/ml.
